(19) United States
(12) Patent Application Publication
(10) Pub. No.: US 2021/0100762 A1
(43) Pub. Date: Apr. 8, 2021

(54) Compositions and methods including reverse thermal gels and pH adjustors

(71) Applicant: Maya Ghosh Rao, Foster City, CA (US)

(72) Inventor: Maya Ghosh Rao, Foster City, CA (US)

(21) Appl. No.: 16/974,023

(22) Filed: Sep. 11, 2020

Related U.S. Application Data
(60) Provisional application No. 62/995,287, filed on Jan. 21, 2020.
(60) Provisional application No. 62/973,241, filed on Sep. 24, 2019.

(51) Int. Cl. A61K 31/194; A61K 9/00; A61K 9/06; A61K 47/34; A61K 31/19

(52) U.S. Cl. CPC A61K 31/194; A61K 9/0034; A61K 31/19; A61K 47/34; A61K 9/06

(57) ABSTRACT

Provided herein are pharmaceutically acceptable compositions suitable for maintaining a low pH, such as a spermicidally effective low pH in a patient's vagina in need thereof. The pharmaceutically acceptable composition comprises a reverse thermal gel. The pharmaceutically acceptable composition further comprises an acidic pH adjustor. Applicators comprising pharmaceutically acceptable compositions provided herein, and therapeutic methods of using the pharmaceutically acceptable compositions provided herein are also provided.

### PRIORITY CLAIM

This Application claims priority to U.S. Provisional application Nos. 62/995,287, filed Jan. 21, 2020, and 62/973,241, filed Sep. 24, 2019, each of which is incorporated herein by reference in its entirety.

### FIELD

Provided herein are pharmaceutically acceptable compositions suitable for maintaining a low pH, such as a spermicidally effective (i.e., a pH effective as a spermicidal) low pH in a patient's vagina in need thereof. The pharmaceutically acceptable composition comprises a reverse thermal gel. The pharmaceutically acceptable composition further comprises an acidic pH adjustor. Applicators comprising pharmaceutically acceptable compositions provided herein, and therapeutic methods of using the pharmaceutically acceptable compositions provided herein are also provided

### BACKGROUND

Teen childbirth is a leading marker of poverty in US. In certain respects, it appears to correlate with one of the worst forms of poverty, persistent poverty. For example, based on US census data in Louisiana, a teen childbirth rate of 40 or more per thousand in a county correlates with persistent poverty in that county.

Yet, since the contraceptive pill of almost 60 years ago, there has been precious little innovation in birth control for women. See, e.g., “Better Birth Control Could Exist, But It Wouldn't Pay for Big Pharma,” Naomi Kresge and Cynthia Koons, Bloomberg Businessweek, Aug. 8, 2019. This has a profound effect not only in women's lifestyle and independence but, due to the consequence of unprotected sexual activity, also on the economy as described above.

There is a need for contraceptive compositions for females, preferably compositions excluding hormones, and more preferably compositions that are available on-demand, and are capable of providing robust short to medium time birth control. Further, based on last sixty years of experience, the supply-side pharmaceutical market is not inclined to address this need.

### SUMMARY

Provided herein are pharmaceutically acceptable compositions suitable for maintaining a low pH, such as a spermicidally effective low pH in a patient's vagina in need thereof. The pharmaceutically acceptable composition comprises a reverse thermal gel. The pharmaceutically acceptable composition further comprises an acidic pH adjustor. Applicators comprising pharmaceutically acceptable compositions provided herein, and therapeutic methods of using the pharmaceutically acceptable compositions provided herein are also provided.

In one aspect, provide herein is a pharmaceutically acceptable composition comprising a reverse thermal gel or a viscosity modulating agent, one or more acidic pH adjustor, and optionally one or more pharmaceutically acceptable excipients. Any pharmaceutically acceptable acid is useful as an acidic pH adjustor. Examples include without limitation, citric, lactic, malonic, glycolic, salicylic, and benzoic acids and monobasic salts of polybasic acids such as citric, tartaric, and phosphoric acid (e.g., and without limitation potassium bitartrate or KH₂PO₄). As used herein, and as is well understood, the solid or semi-solid (jelly like) stage of a colloidal dispersion is called a gel; the fluid state of a colloidal solution is called the sol, also referred to herein as a solution. A gel contains a liquid in which particles, such as those too small to be seen in an ordinary optical microscope are either dispersed or arranged in a fine network. A gel may be notably elastic and jelly like. Gels are typically colloids (aggregates of fine particles, as described above, dispersed in a continuous medium) in which the liquid medium has become viscous enough to behave more or less as a solid. Accordingly, a gelator is a substance, which when mixed in a media, such as an aqueous media, provides a mixture that is capable of forming a gel. For example, and without limitation, ESHU, PEG-PACU, acylated PCLA-PEG-PCLA, PLGA-PEG-PLGA, poloxamers, such as poloxamers 407, 188, and the likes, are gelators useful in certain aspects and embodiments of the invention provided herein. The abbreviations ESHU, PEG, PACU, PLGA, PCLA and such other terms used herein are explained herein below, and/or are well known to a skilled artisan. In certain embodiments, the gelators utilized herein are biodegradable. In some embodiments, the biodegradable gelators comprise ester or carbonate linkages. In some embodiments, the biodegradable gelators utilized herein biodegrade at least 2-10 times faster than a poloxamer. Methods of determining biodegradability is well known. See, e.g., “Biodegradability of Polymers: Regulations and Methods for Testing,” Part 10. General Aspects and Special Applications, Rolf-Joachim Muller, Biopolymers Online, Wiley VCH, Copyright © 2005 by Wiley-VCH (incorporated herein by reference).

In some embodiments, about 20% to about 50%, or about 20% to about 40%, or about 30% to about 40%, or about 20% by weight of the composition is the gelator.

In another aspect, provided herein is an applicator comprising a pharmaceutically acceptable composition provided herein. In one embodiment, the applicator is suitable for intra-vaginal application.

In another aspect, provided herein is a method of maintaining intravaginal pH at a pH of about 3.0-about 5.0, comprising administering an effective amount of the pharmaceutically acceptable composition provided herein vaginally thereby maintaining an intravaginal pH of about 3.0-about 5.0.

In another aspect, provided herein is a method of female contraception, comprising administering a therapeutically effective amount of the pharmaceutically acceptable composition provided herein vaginally to a female in need thereof.

In another aspect, provided herein is a method of prophylaxis or treatment of vaginal infection or urinary tract infection, comprising administering a therapeutically effective amount of a pharmaceutically acceptable composition provided herein to a patient in need thereof.

In another aspect, the pharmaceutically acceptable composition provided herein is for female contraception or prophylaxis or treatment of vaginal infection or urinary tract infection.

In one embodiment, the administration is performed once every day to once every three months. In one embodiment, the administration is performed once every day, once every other day, once every week, once every month, or once every three months.

In another aspect provided herein is a pharmaceutically acceptable composition comprising an

(a) A-B-A type block copolymer, wherein A comprises a polyethylene glycol, and B comprises a polyurethane;

a copolymer, preferably a block copolymer comprising PEG and a polyester or a copolyester of one or more omega hydroxy carboxylic acids, such as without limitation, B-A-B type block copolymer, wherein A comprises polyethylene glycol (PEG) or a mono alkylated PEG (such as methoxy PEG or mPEG), and B comprises PLGA or a lactic acid-glycolic copolymer, or A comprises a lactic acid polymer, or A comprises a lactic acid-epsilon hydroxy hexanoic acid (or caprolactone) copolymer, or A comprises an epsilon hydroxy hexanoic acid (or caprolactone) copolymer;

an amine-functionalized ABA block copolymer, polyethylene glycol)-poly(serinol hexamethylene urethane), or ESHU, wherein the copolymer comprises a hydrophobic block (B): poly(serinol hexamethylene urethane) and a hydrophilic block (A): poly(ethylene glycol) (ESHU);

a (PEG-PACU) wherein the polyurethane excludes an amine functionality which is protonated at pH 3-5;

a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-functionalized polymer, acrylamide-based copolymer, and acrylonitrile-based copolymer compounds;

a copolymer having at least a hydrophobic segment and at least a hydrophilic segment, wherein the hydrophobic segment(s) and the hydrophilic segment(s) are capable of self assembly into a micelle;

or a copolymer having a hydrophobic segment and one or more hydrophilic segments, wherein the hydrophobic segment and the one or more hydrophilic segments are capable of self assembly into a micelle;

(b) one or more acidic pH adjustor, and

(c) optionally one or more pharmaceutically acceptable excipients.

In one embodiment, the reverse thermal gel comprises a copolymer having at least one hydrophobic segment and at least one hydrophilic segment, wherein the hydrophobic segment(s) and the hydrophilic segment(s) are capable of self assembly into a micelle. In one embodiment, the reverse thermal gel comprises a copolymer, such as, e.g., a copolymer having a hydrophobic segment and one or more hydrophilic segments, as described in US Pat App. No. 2013/0129663 (incorporated herein by reference).

In one embodiment, the pharmaceutically acceptable composition comprises a B-A-B type block copolymer, wherein A comprises polyethylene glycol, and B comprises PLGA or a lactic acid-glycolic acid copolymer. In another embodiment, the B-A-B type block copolymer, wherein A comprises polyethylene glycol, and B comprises PLGA or a lactic acid-glycolic copolymer, is water soluble. In another embodiment, the B-A-B type block copolymer, wherein A comprises polyethylene glycol, and B comprises PLGA or a lactic acid-glycolic copolymer, forms a reverse thermal gel, e.g., in an aqueous medium. In another embodiment, the B-A-B type block copolymer, wherein A comprises polyethylene glycol, and B comprises PLGA or a lactic acid-glycolic copolymer, forms an aqueous sol at about 20 to about 30° C. In another embodiment, the B-A-B type block copolymer, wherein A comprises polyethylene glycol, and B comprises PLGA or a lactic acid-glycolic copolymer, forms a gel at about 35 to about 40° C.

In some embodiments, the A block of the block polymer utilized herein is polyethylene glycol (PEG, polyethylene oxide, or PEO) of molecular weight of about 200 to about 2000. In some embodiments, the A block of the block polymer utilized herein is polyethylene glycol (PEG, polyethylene oxide, or PEO) of molecular weight of about 200 to about 1700. As used herein, molecular weight refers to weight average (M_{W}) or number average (M_{N}) molecular weight. In one embodiment, the molecular weight is weight average molecular weight. In another embodiment, the molecular weight is number average molecular weight In some embodiments, the PEG is of molecular weight of about 400. In some embodiments, the PEG is of molecular weight of about 600. In some embodiments, the PEG is of molecular weight of about 800. In some embodiments, the PEG is of molecular weight of about 1000. In some embodiments, the PEG is of molecular weight of about 1200. In some embodiments, the PEG is of molecular weight of about 1500. In some embodiments, the PEG is of molecular weight of about 2000.

In some embodiments, the PLGA block of the block polymer utilized herein is of molecular weight of about 200-about 2000. In some embodiments, the PLGA block of the block polymer utilized herein is of molecular weight of about 200-about 1700. In some embodiments, the PLGA block of the block polymer utilized herein is of molecular weight of about 200. In some embodiments, the PLGA block of the block polymer utilized herein is of molecular weight of about 400. In some embodiments, the PLGA block of the block polymer utilized herein is of molecular weight of about 500. In some embodiments, the PLGA block of the block polymer utilized herein is of molecular weight of about 600. In some embodiments, the PLGA block of the block polymer utilized herein is of molecular weight of about 800. In some embodiments, the PLGA block of the block polymer utilized herein is of molecular weight of about 1000. In some embodiments, the PLGA block of the block polymer utilized herein is of molecular weight of about 1100. In some embodiments, the PLGA block of the block polymer utilized herein is of molecular weight of about 1200. In some embodiments, the PLGA block of the block polymer utilized herein is of molecular weight of about 1300. In some embodiments, the PLGA block of the block polymer utilized herein is of molecular weight of about 1500. In some embodiments, the PLGA block of the block polymer utilized herein is of molecular weight of about 1600. In some embodiments, the PLGA block of the block polymer utilized herein is of molecular weight of about 1700. In some embodiments, the PLGA block of the block polymer utilized herein is of molecular weight of about 2000.

In the PLGA containing thermosensitive polymers utilized herein, in some embodiments, the ratio of lactic acid (LA): glycolic acid (GA) is about 1:1 to about 15:1 weight by weight (w/w). In some embodiments, the ratio of LA:GA is about 1:1. In some embodiments, the ratio of LA:GA is about 2:1. In some embodiments, the ratio of LA:GA is about 3:1. In some embodiments, the ratio of LA:GA is about 6:1. In some embodiments, the ratio of LA:GA is about 15:1.

In some embodiments, the polylactic acid-caprolactone (PCLA) block of the acyl capped PCLA-PEG-PCLA block polymer utilized herein is of molecular weight of about 200-about 2000. In some embodiments, the PCLA block of the block polymer utilized herein is of molecular weight of about 200. In some embodiments, the PCLA block of the block polymer utilized herein is of molecular weight of about 400. In some embodiments, the PCLA block of the block polymer utilized herein is of molecular weight of about 500. In some embodiments, the PCLA block of the block polymer utilized herein is of molecular weight of about 600. In some embodiments, the PCLA block of the block polymer utilized herein is of molecular weight of about 800. In some embodiments, the PCLA block of the block polymer utilized herein is of molecular weight of about 1000. In some embodiments, the PCLA block of the block polymer utilized herein is of molecular weight of about 1100. In some embodiments, the PCLA block of the block polymer utilized herein is of molecular weight of about 1300. In some embodiments, the PCLA block of the block polymer utilized herein is of molecular weight of about 1500. In some embodiments, the PCLA block of the block polymer utilized herein is of molecular weight of about 2000.

In some embodiments, the acyl moiety of the acy capped PCLA-PEG-PCLA block polymer is —COR⁵. In one embodiment, R⁵ is lower alkyl. In one embodiment, R⁵ is C₁-C₆ alkyl. In one embodiment, R⁵ is C₃-C₆ cycloalkyl. In one embodiment, R⁵ is C₃-C₄ cycloalkyl. In some embodiments, the alkyl and cycloalkyl groups are substituted with substituents that do not negatively impact the reverse thermal gelling property. In some embodiments, the substituent is fluoro, alkoxy such as C₁-C₆ alkoxy, and the likes. In some embodiments, the acyl moiety of the acy capped PCLA-PEG-PCLA block polymer is an acetyl group. In some embodiments, the acyl moiety of the acyl capped PCLA-PEG-PCLA block polymer is a propyl group. In some embodiments, the acyl moiety of the acyl capped PCLA-PEG-PCLA block polymer is a butyl group. Certain acyl capped reverse thermogel PCLA-PEG-PCLA polymers are described in U.S. Pat. No. 9,795,679 (incorporated by reference), and one of skilled in the art can adapt or modify such polymers and methods described therein.

Compositions and methods provided herein comprising biodegradable polymers such as without limitation PLGA-PEG-PLGA or acyl capped PCLA-PEG-PCLA, include certain advantages. Without being bound by theory, the biodegradability allows for more efficient disposal, despite lower erosion, after application of the reverse thermal gel in accordance with the present invention. Further, without being bound by theory, the biodegradation of the polyester polymers, such as PLGA-PEG-PLGA and the likes utilized herein, provides organic acids such as lactic, glycolic, and such other acids, which are capable of lowering the intravaginal pH or maintaining a low intravaginal pH, and acting as a spermicidal and a contraceptive as provided herein.

In some aspects, the compositions and methods provided herein exclude a separate pH adjustor.

Non-limiting and illustrative examples of thermosensitive reverse thermal gelling PEG polyester polymers useful according to the compositions and methods provided herein include the following block copolymers:
Poly(DL-lactide)-Poly(ethylene glycol)-Poly(DL-lactide)
PLA-PEG-PLA (1500-1500-1500 Da)

Poly(lactide-co-caprolactone)-b-Poly(ethylene glycol)-b-Poly(lactide-co-caprolactone)

PLCL-PEG-PLCL (1,700-1,500-1,700 Da), 90:10 caprolactone (CL): lactide (LA)

Poly(DL-lactide)-Poly(ethylene glycol)-Poly(DL-lactide), where “DL” refers to the stereochemistry of the lactide

PLA-PEG-PLA (1,750-1,500-1,750 Da)

Poly(lactide-co-glycolide)-poly(ethylene glycol)-poly(lactide-co-glycolide)

Synonym: Poly(lactic-co-glycolic acid)-Poly(ethylene glycol)-Poly(lactic-co-glycolic acid)

PLGA-PEG-PLGA 15:1 lactic acid (LA): glycolic acid (GA), 1,750-1,500-1,750 Da

Poly(lactide-co-glycolide)-b-poly(ethyelen glycol)-b-poly(lactide-co-glycolide)

PLGA-PEG-PLGA 1,500:1,500:1,500 Da, LA:GA 95:5 (20:1)

Poly(lactide-co-glycolide)-Poly(ethylene glycol)-Poly(lactide-co-glycolide)

Synonym: Polylactic-co-glycolic acid)-b-Poly(ethylene glycol)-b-Poly(lactic-co-glycolic acid)

PLGA-PEG-PLGA (750:1,500:750 Da, LA:GA 15:1)

Poly(lactide-co-glycolide)-Poly(ethylene glycol)-Poly(lactide-co-glycolide)

Synonym: Polylactic-co-glycolic acid)-b-Polyethylene glycol)-b-Polylacticco-glycolic acid)

PLGA-PEG-PLGA (1,000:1,000:1,000 Da) (LA:GA 6:1)

Poly(lactide-co-glycolide)-Poly(ethylene glycol)-Poly(lactide-co-glycolide)

Synonym: Polylactic-co-glycolic acid)-Poly(ethylene glycol)-Poly(lactic-co-glycolic acid)

PLGA-PEG-PLGA (1,000:1,000:1,000 Da), LA:GA 15:1)

Poly(lactide-co-caprolactone)-Poly(ethylene glycol)-Poly(lactide-co-caprolactone)

Synonym: Poly(lactide-co-caprolactone)-b-Poly(ethylene glycol)-b-Poly(lactide-cocaprolactone)

PLCL-PEG-PLCL (1600-1500-1600 Da), 75:25 CL:LA publications

Poly(lactide-co-caprolactone)-Poly(ethylene glycol)-Poly(lactide-co-caprolactone)

Synonym: Poly(lactide-co-caprolactone)-b-Poly(ethylene glycol)-b-Poly(lactide-cocaprolactone)

PLCL-PEG-PLCL (1700-1500-1700 Da), 60:40 CL:LA)

Poly(DL-lactide)-Poly(ethylene glycol)-Poly(DL-lactide),

P(DL)LA-PEG-P(DL)LA (1700-1500-1700Da)

Poly(lactide-co-glycolide)-Poly(ethylene glycol)-Poly(lactide-co-glycolide)

(or Polylactic-co-glycolic acid)-Poly(ethylene glycol)-Poly(lactic-co-glycolic acid))

PLGA-PEG-PLGA (1700-1500-1700Da) (LA:GA 15:1(94%/6% LA/GA) (w:w))

Poly(lactide-co-glycolide)-Poly(ethylene glycol)-Poly(lactide-co-glycolide)

(or Poly(lactic-co-glycolic acid)-b-Poly(ethylene glycol)-b-Poly(lactic-co-glycolic acid)) PLGA-PEG-PLGA (1,500:1,500:1,500 Da), 6:1 LA:GA (86%/14% LA/GA)

Poly(lactide-co-glycolide)-Poly(ethylene glycol)-Poly(lactide-co-glycolide)

(or Poly(lactic-co-glycolic acid)-b-Polyethylene glycol)-b-Poly(lactic-co-glycolic acid))

PLGA-PEG-PLGA (1,700:1,500:1,700 Da), 3:1 LA:GA

Poly(L-lactide-co-glycolide)-poly(ethylene glycol)-poly(L-lactide-co-glycolide)

PLLGA-PEG-PLLGA LG 75:25 (w:w), (1,100:1,000:1,100 Da)

Poly(lactide-co-glycolide)-poly(ethylene glycol)-poly(lactide-co-glycolide)

(or Polylactic-co-glycolic acid)-b-Polyethylene glycol)-b-Poly(lactie-co-glycolic acid))

PLGA-PEG-PLGA LG 75:25, (1,600:1,500:1,600 Da)

Poly(lactide-co-glycolide)-poly(ethylene glycol)-poly(lactide-co-glycolide)

(or Poly(lactic-co-glycolic acid)-b-Polyethylene glycol)-b-Poly(lactic-co-glycolic acid))

PLGA-PEG-PLGA LG 50:50 (w:w), (1,400:1,500:1,400 Da)

Poly(D,L-lactide)-poly(ethylene glycol)-poly(D,L-lactide)

PLA-PEG-PLA (1,000:1,000:1,000 Da)

Poly(caprolactone)-b-poly(ethylene glycol)-b-poly(caprolactone)

PCL-PEG-PCL (1,000:1,000:1,000 Da)

Methoxy poly(ethylene glycol)-b-poly(caprolactone)

(or Methoxy Poly(ethylene glycol)-b-Polycaprolactone)

mPEG-PCL (750:2,500 Da)

Poly(lactide-co-glycolide)-poly(ethylene glycol)-poly(lactide-co-glycolide)

(or Poly(lactic-co-glycolic acid)-b-Poly(ethylene glycol)-b-Poly(lactic-co-glycolic acid))

PLGA-PEG-PLGA LG 75:25 (w:w) (1,100:1,000:1,100 Da), and

Poly(lactide-co-glycolide)-poly(ethylene glycol)-poly(lactide-co-glycolide)

(or Polylactic co-glycolic acid)-b-Poly(ethylene glycol)-b-Polylactic-co-glycolic acid))

PLGA-PEG-PLGA LG 50:50 (w:w) (1,000:1,000:1,000 Da).

As is apparent, polylactide or polyglycolide and poly lactic acid or poly glycolic acid are used interchangeably in the examples above. The ratios of lactide (or lactic acid) and glycolide (or glycolic acid) within PLGA are weight by weight ratios.

These and other classes of reverse thermogels suitable in accordance with the present invention are described e.g. in Sing Shy Liow et al., and ACS Biomater. Sci Eng., 2016, 2, 295-316 and Polymers (Basel), 2011 Sep. 1; 3(3): 1377-1397; “Poly Lactic-co-Glycolic Acid (PLGA) as Biodegradable Controlled Drug Delivery Carrier,” Hirenkumar K. Makadial and Steven J. Siegel, (incorporated herein by reference) and can be adapted or modified by the skilled artisan to practice the present invention.

Many of the PEG polyester block polymers utilized herein are commercially available, e.g., from Sigma Aldrich or from Akina Inc. (https://akinainc.com/polyscitech/products/polyvivo/catalogue.php#BlockCopolymers). The PEG polyesters utilized herein can be prepared by polymerizing one or more of a lactide, a glycolide, a lactone such as epsilon caprolactone, under conditions well known in the art or upon modifying them based on the skill of one of skilled in the art. Various Lewis acids are useful such as e.g., tin based Lewis acids. Base catalyzed polymerizations are also contemplated based e.g. on using DBU. See, Haitao Qian et al., Macromolecules, 2011, 44(18): 7132-7140 (incorporated herein by reference).

In some embodiments, the copolymer utilized herein, such as the triblock copolymer, is water-soluble. In some embodiments, the copolymer utilized herein, such as the triblock copolymer is biodegradable. The gel formation from a composition, such as a polymer composition, provided herein is observed by various art known methods, e.g., and without limitation by injecting the polymer composition, such as a sol or a solution subcutaneously injected into rats or chicken, and observing depot formation. In some embodiments, the polymeric gel depots last for two weeks in vivo.

The compositions provided and utilized here are contemplated to be with and without an additional spermicide. A non limiting example of a spermicide is nonoxynol-9. In one embodiment, the compositions and methods provided herein exclude a spermicide such as nonoxynol-9.

In certain preferred embodiments, the compositions provided herein are easy to apply, and e.g. exist in a sol or solution state before and during application, but once intravaginal, undergo a transition to an immobile, or semisolid, or a gel state, and are capable of adhering to the intravaginal surface and provide spermicidal capability.

A reverse thermal gel containing 1 part by weight of a poloxamer, such as poloxamer 188, and 2 parts of water was prepared, and acidified to a pH of about 3 -about 4 with citric acid. The composition was a sol at room temperature and was a gel at elevated temperatures.

Other reverse thermal gels useful according to the formulations and methods provided herein, including, without limitation, those comprising one or more PEG moieties or blocks, and one or more hydrophobic moieities or blocks, such as polyhydroxy alkanoates, including co-polyhydroxy alkanoates, are described e.g. in:

Biomaterials. 2011 January; 32(3): 777-786, “A functionalizable reverse thermal gel based on a polyurethaneJPEG block copolymer,” Daewon Park, Wei Wu, and Yadong Wang;

Eur. J. Pharm. Biopharm., 2008; 68(1): 34-45, “Thermoresponsive hydrogels in biomedical applications—a review,” Leda Klouda and Antonios G. Mikos;

J. Mater. Chem., 2009,19, 5891-5905, “Reverse thermogelling biodegradable polymer aqueous solutions,” Min Kyung Joo, Min Hee Park, Bo Gyu Choia and Byeongmoon Jeong;

“Supramolecular Hydrogels with Reverse Thermal Gelation Properties from (Oligo)tyrosine Containing Block Copolymers,” Jin Huang, Conn L. Hastings, Garry P. Duffy, Helena M. Kelly, Jaclyn Raebum, Dave J. Adams, Andreas Heise, Biomacromolecules, 2013, 141, 200-206; and

ACS Biomater. Sci. Eng. 2016, 2, 295-316, “Thermogels: In Situ Gelling Biomaterial,” Sing Shy Liow, Qingqing Dou, Dan Kai, Anis Abdul Karim, Kangyi Zhang, Fujian Xu, and Xian Jun Loh. Each of which is incorporated herein by reference.

### DETAILED DESCRIPTION

The practice of the present invention includes the use of conventional techniques of organic chemistry, pharmacology, and chemistry manufacturing and controls, which are within the skill of the art.

### Definitions

In this specification and in the claims that follow, reference will be made to a number of terms that have the meanings below. All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about” Reagents described herein are exemplary and equivalents of such may be known in the art.

The singular form “a”, “an”, and “the” includes plural references unless the context clearly dictates otherwise.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Patient” or “subject” refers to mammals, particularly female humans, and so includes animals of veterinary and research interest, such as simians, cattle, horses, dogs, cats, and rodents.

“Pharmaceutically acceptable” refers to safe, non-toxic, and stable for the intended pharmaceutical ministration.

“Therapeutically effective amount” of a drug or an agent refers to an amount of the drug or the agent that, when administered to a patient needing to maintain a low intravaginal pH, will have the intended therapeutic effect. A therapeutic effect does not necessarily occur by administration of one dose, and may occur only ager administration of a series of doses. Thus, a therapeutically effective amount may be administered in one or more administrations.

“Treating” or “treatment of” a condition or patient refers to taking steps to obtain beneficial or desired results, including clinical results. For purposes of this invention, beneficial or desired clinical results include, but are not limited to, maintaining a low intravaginal pH or other beneficial results.

“Excipient” refers to an inactive substance that serves, e.g., as a vehicle or medium for a drug or other active substance. Excipients include, without limitation, viscosity modulating agents, coloring agents, preservatives, fillers, and fragrance.

### Compositions and Methods

In one aspect, provide herein is a pharmaceutically acceptable composition comprising a reverse thermal gel, one or more acidic pH adjustor, and optionally one or more pharmaceutically acceptable excipients.

In one embodiment, the one or more acidic pH adjustor maintains a pH of about 3-about 5. In one embodiment, the one or more acidic pH adjustor maintains a pH of about 3-about 4. In one embodiment, the one or more acidic pH adjustor maintains a pH of about 3. In one embodiment, the one or more acidic pH adjustor maintains a pH of about 4.

In some embodiments, the pharmaceutically acceptable compositions provided herein include one or more poloxamers. In some embodiments, the pharmaceutically acceptable compositions provided herein include one or more poloxamers exhibit reverse thermal gelling such as the composition is a sol at about 25° C. and forms a gel as the temperature increases to 35-40° C. Examples of poloxamers include poloxamer 407, 188, 184, and the likes, and mixtures thereof. In one embodiment, the mixture comprises poloxamers 407 and 188. In one embodiment, the poloxamer or the mixture of poloxamers provide a composition, which, preferably at a pH of about 3-5, is a liquid at room temperature, such as at about 20 about 25° C., or about 25° C. In one embodiment, the poloxamer or the mixture of poloxamers provide a composition, which, preferably at a pH of about 3-5, is a gel at intravaginal temperature, such as at about 30°-about 40° C. or about 35°-about 40° C. In one embodiment, the poloxamer or the mixture of poloxamers provide a composition, which, preferably at a pH of about 3-5, is a gel at a temperature at about 35-40° C. In one embodiment, the pharmaceutical composition provided herein comprises about 17% to about 20% per weight of poloxamer 407. In one embodiment, the pharmaceutical composition provided herein comprises about 3% to about 15% per weight of poloxamer 188. In one embodiment, the pharmaceutical composition provided herein comprises about 17% to about 20% per weight of poloxamer 407 and about 3%-about 15% per weight of poloxamer 188, wherein the pharmaceutical composition is liquid or sol at about 4° C.-about 32° C. and forms a gel at about 37° C. In one embodiment, the gel is a semi solid. In one embodiment, the gel formation is determined by an inverted test tube method.

In another embodiment, the reverse thermal gel excludes a poloxamer.

In one embodiment, the reverse thermal gel comprises a copolymer, such as, e.g., a copolymer having a hydrophobic segment and one or more hydrophilic segments, as described in US Pat App. No. 2013/0129663 (incorporated herein by reference). In one embodiment, the copolymer is of structure:

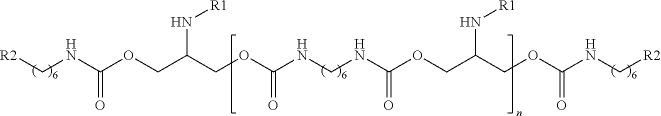

wherein R1 is H or an amine protective group, R2 is isocyanate or —NC(O)—PEG and n is greater than 5, for example and without limitation, 8-30, 8-25 or 18-30.

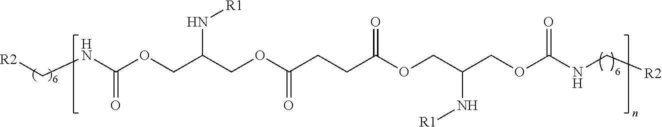

wherein R1 and R2 and n are defined as above. Each hexamethylene moiety —(CH₂)₆— as shown above, can be modified, independent of each other, in accordance with the invention provided herein so that the number of methylene is more or less than 6, such as 4, 5, 7-10, or more or less.

In one embodiment, the pharmaceutical composition excludes a poloxamer.

In one embodiment, the reverse thermal gel comprises a triblock copolymer comprising an acylated PEG, L-lactide, and ε-caprolactone or acyl-capped poly(ε-caprolactone-co-lactide)-b-poly(ethylene glycol)-b-poly(ε-caprolactone-co-lactide) (PCLA-PEG-PCLA) triblock copolymers. In one embodiment, the reverse thermal gel comprising an acylated triblock copolymer comprising PEG, L-lactide, and ε-caprolactone polymer has the structure of formula: CH₃—[CH₂]ₘ—COO—[[(CH₂)5-COO]y-[CH(CH₃)COO]xHCH₂—CH₂—O]n-[[-CO—CH(CH₃)—O]x-[CO—(CH₂)5-]y]-OCO—[CH₂]m-CH₃

where m, y, x, and n are independently integers denoting repeat units of the corresponding moieties. See, e.g., Seongbong Jo, Tm Kim, and Sung Wan Kim, “Reverse Thermal Gelation of Aliphatically Modified Biodegradable Triblock Copolymers,” Macromolecular Bioscience, Volume 6, Issue 11, Nov. 9, 2006, pages 923-928; Biomaterials, 2006, 27(9):1718-27, “PEO-PPO-PEO-based poly(ether ester urethane)s as degradable reverse thermo-responsive multiblock copolymers,” Cohn D, Lando G, Sosnik A, Garry S, and Levi A; Audrey Petit et al. “Effect of Polymer Composition on Rheological and Degradation Properties of Temperature-Responsive Gelling Systems Composed of Acyl-Capped PCLA-PEG-PCLA,” Biomacromolecules, 2013, 149: 3172-3182; Petit et al., Biomaterials, Volume 53, Pages 426-436; and Eur J. Pharm. Biopharm. 2008, 68(1): 34-45, “Thermoresponsive hydrogels in biomedical applications,” Leda Klouda and Antoni os G. Mikos (each of which is incorporated herein by reference). ESHU is synthesized according to Biomaterials. 2011, 32(3): 777-786, “A functionalizable reverse thermal gel based on a polyurethane/PEG block copolymer,” Daewon Park, Wei Wu, and Yadong Wang, (incorporated herein by reference). See also Macromol. Biosci., 2013, 13(4): 464-469, “An Anti-angiogenic Reverse Thermal Gel as a Drug-Delivery System for Age-Related Wet Macular Degeneration,” Daewon Par, Veeral Shah, Britta M. Rauck, Thomas R. Friberg, and Yadong Wang (incorporated herein by reference).

In another embodiment, the reverse thermal gel is: an A-B-A type block copolymer. In another embodiment, the polymer A comprises a polyethylene glycol. In another embodiment, the polymer B comprises a polyurethane In another embodiment, the reverse thermal gel comprises ESHU. In another embodiment, the reverse thermal comprises a polyethylene glycol-poly (amino carbonate urethane) (PEG-PACU, see KR101815780, incorporated herein by reference). In another embodiment, the polyurethane excludes an amine functionality which is protonated at pH 3-5.

In another embodiment, the reverse thermal gel comprises a copolymer having a hydrophobic segment and one or more hydrophilic segments. In another embodiment, the hydrophobic segments and the one or more hydrophilic segments are capable of self assembly into a micelle. In another embodiment, the hydrophilic segment comprises polyethylene glycol. In another embodiment, the hydrophilic segment is derived from polyethylene glycol. In another embodiment, the hydrophobic segment comprises or is derived from poly(hexamethylene-alt-serinol) (PHS). In one embodiment, the hydrophobic segment has a molecular weight of about 75,000 to about 200,000 Da.

In another embodiment, the reverse thermal gel comprises a grafted copolymer on a backbone polymer. In another embodiment, the backbone polymer comprises poly[hexamethylene-alt-(serinol urea)] (PHSU). In another embodiment, the graft copolymer is poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-functionalized polymer, acrylamide-based copolymer, or acrylonitrile-based copolymer compounds.

In another embodiment, the backbone polymer comprises esterified poly[hexamethylene-alt-(serinol urea)]. In another embodiment, the backbone polymer has a molecular weight of about 2,000 Da to about 50,000 Da. In another embodiment, the graft polymer has a molecular weight between about 2,000 Da and 50,000 Da.

In another embodiment, the backbone polymer comprises a diol compound, wherein the diol compound comprises an amino-substituted or N-substituted serinol in which the nitrogen atom is attached to one of a hydrogen, an amine protective group or an active agent.

In another embodiment, the graft polymer comprises poly(N-isopropylacrylamide). In another embodiment, the graft polymer has a molecular weight of about 2,000 Da to about 100,000 Da

In another embodiment, the diol is an amino-substituted or N-substituted serinol, wherein the N is attached to one of a hydrogen, an amine protective group, or an active agent. In another embodiment, the N of the N-substituted serinol is —NHR in which R is a protective group, such as an amino protective group. In another embodiment, R is selected from the group consisting of carbobenzyloxy; p-methoxybenzyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl. In another embodiment, R is tert-butyloxycarbonyl.

In another embodiment, the diol comprises one or more ester groups. In another embodiment, the diol is a reaction product of a cyclic anhydride and a diol comprising one or more pendant active groups, blocked active groups or active agents. In another embodiment, the diol is the reaction product of succinic anhydride and the diol is an N-substituted serinol in which the N is attached to one of a hydrogen, a protective group, or an active agent In another embodiment, the diol comprises a pendant amino group or an amine.

In another embodiment, the reverse thermal gel comprises a urethane, which comprises as a diisocyanate hexamethylene diisocyanate (1,6-diisocyanatohexane).

In another embodiment, the pharmaceutically acceptable composition is a sol or a solution at about 4° C. In another embodiment, the pharmaceutically acceptable composition is a sol or a solution at about 5 -about 10° C. In another embodiment, the pharmaceutically acceptable composition is a sol or a solution at room temperature. In another embodiment, the pharmaceutically acceptable composition is a gel at about 35° C.-about 40° C. under physiological conditions. In another embodiment, the pharmaceutically acceptable composition is a gel at about 35° C.-about 40° C. under physiological conditions as determined by an inverted test tube method. An inverted test tube method is a well known method for determining sol to gel transition temperature or T_{gel}, and is described in the examples section. In another embodiment, the pharmaceutically acceptable composition is a gel at about 37° C. under physiological conditions. In one embodiment, the T_{gel} is determined by an inverted test tube method.

In another aspect, provided herein is an applicator comprising a pharmaceutically acceptable composition provided herein. In another embodiment, the applicator is a syringe, a tube, a dropper, or a sprayer.

In another aspect, provided herein is a method of maintaining intravaginal pH at a pH of about 3.0-about 5.0, comprising administering an effective amount of the pharmaceutically acceptable composition provided herein vaginally thereby maintaining an intravaginal pH of about 3.0-about 5.0.

In another aspect, provided herein is a method of female contraception, comprising administering a therapeutically effective amount of the pharmaceutically acceptable composition provided herein vaginally to a female in need thereof.

In another aspect, provided herein is a method of prophylaxis or treatment of vaginal infection or urinary tract infection, comprising administering a therapeutically effective amount of a pharmaceutically acceptable composition provided herein to a patient in need thereof.

In another aspect, the pharmaceutically acceptable composition provided herein is for female contraception or prophylaxis or treatment of vaginal infection or urinary tract infection.

In one embodiment, the method is a method of prophylaxis. In another embodiment, the method is a method of treatment.

In another aspect provided herein is a pharmaceutically acceptable composition comprising an

(a) A-B-A type block copolymer, wherein A comprises a polyethylene glycol, and B comprises a polyurethane;

an (ESHU);

a (PEG-PACU) wherein the polyurethane excludes a amine functionality which is protonated at pH 3-5;

a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-functionalized polymer, acrylamide-based copolymer, and acrylonitrile-based copolymer compounds; or

a copolymer having a hydrophobic segment and one or more hydrophilic segments, wherein the hydrophobic segment and the one or more hydrophilic segments are capable of self assembly into a micelle;

(b) one or more acidic pH adjustor, and

(c) optionally one or more pharmaceutically acceptable excipients.

In one embodiment, the composition excludes a poloxamer.

In another embodiment, the composition comprises a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-functionalized polymer, acrylamide-based copolymer, and acrylonitrile-based copolymer compounds.

In another embodiment, the backbone polymer comprises esterified poly[hexamethylene-alt-(serinol; urea)].

In another embodiment, the backbone polymer comprises a diol compound, wherein the diol compound comprises an amino-substituted or N-substituted serinol in which the nitrogen atom is attached to one of a hydrogen, a protective group or an active agent.

In another embodiment, the graft polymer comprises poly(N-isopropylacrylamide).

In another embodiment, the composition comprises: a copolymer comprising a hydrophobic segment and one or more hydrophilic segments wherein the hydrophobic segment and one or more hydrophilic segments self-assemble into a micelle.

In another embodiment, the composition comprises: a copolymer comprising a hydrophobic segment and one or more hydrophilic segments wherein the hydrophobic segment and one or more hydrophilic segments self-assemble into a micelle. In another embodiment, the hydrophilic segment is derived from polyethylene glycol. In another embodiment, the hydrophobic segment comprises or is derived from poly(hexamethylene-alt-serinol) (PHS). In another embodiment, and the hydrophobic segment has a molecular weight of about 75,000 to about 200,000 Da.

In another embodiment, the diol is an amino-substituted or N-substituted serinol, wherein the N is attached to one of a hydrogen, a protective group, or an active agent.

In another embodiment, the N of the N-substituted serinol is —NHR in which R is a protective group.

In another embodiment, R is selected from the group consisting of carbobenzyloxy; p-methoxybenzyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl.

In another embodiment, R is tert-butyloxycarbonyl.

In another embodiment, the diol comprises one or more ester groups.

In another embodiment, the diol is a reaction product of a cyclic anhydride and a diol comprising one or more pendant active groups, blocked active groups or active agents.

In another embodiment, the diol is the reaction product of succinic anhydride and the diol is an N-substituted serinol in which the N is attached to one of a hydrogen, a protective group, or an active agent.

In another embodiment, the diol comprises a pendant amino group or an amine.

In another embodiment, the diisocyanate is hexamethylene diisocyanate (1,6-diisocyanatohexane).

In another embodiment, the pharmaceutically acceptable composition is a sol or a solution at room temperature, and form a gel at about 35° C.-about 40° C., such as at about 37° C. under physiological conditions.

In another aspect, provided herein is an applicator such as a syringe, a tube, a dropper, comprising the pharmaceutically acceptable composition provided herein.

In another aspect, provided herein is a method of maintaining intravaginal pH at a pH of about 3.0-about 5.0, comprising administering vaginally an effective amount of a composition provided herein thereby maintaining an intravaginal pH of about 3.0-about 5.0.

In another aspect, provided herein is a method of female contraception, comprising administering a therapeutically effective amount of the pharmaceutically provided herein vaginally to a female in need thereof.

In another aspect, provided herein is a method of prophylaxis or treatment of vaginal infection or urinary tract infection, comprising administering a therapeutically effective amount of the pharmaceutically acceptable composition provided herein.

In another aspect, the pharmaceutically acceptable composition provided herein is for female contraception or for prophylaxis or treatment of vaginal infection or urinary tract infection.

Reverse thermal gels for other purposes are reported e.g. in US patent app. Pub. No. US 2017/0340756; U.S. Pat. No. 7,018,645; U.S. Pat. No. 9,901,554; and V. H. Giang Phan et al., “Poly(amino carbonate urethane)-based biodegradable, temperature and pH-sensitive injectable hydrogels for sustained human growth hormone delivery,” Sci Rep. 2016; 6: 29978, (each incorporated herein by reference), which can be modified by a skilled artisan in accordance with the present disclosure.

The compositions and methods provided herein include the following enumerated as 1-49.
1. A pharmaceutically acceptable composition comprising a reverse thermal gel, one or more acidic pH adjustor, and optionally one or more pharmaceutically acceptable excipients.
2. The composition of 1, wherein the one or more acidic pH adjustor maintains a pH of about 3-5.
3. The composition of 1, wherein the reverse thermal gel is: an A-B-A type block copolymer, wherein A comprises a polyethylene glycol, and B comprises a polyurethane; a B-A-B type block copolymer, wherein A comprises polyethylene glycol, and B comprises a polyhdroxyalkanoate, such as a co polymer of 2 hydroxyalkanoates, preferably, PLGA or a lactic acid-glycolic copolymer, an (ESHU); or a (PEG-PACU) wherein the polyurethane excludes an amine functionality which is protonated at pH 3-5.
4. The composition of 1, wherein the reverse thermal gel comprises a copolymer having a hydrophobic segment and one or more hydrophilic segments.
5. The composition of 5, wherein the hydrophilic segment comprises or is derived from polyethylene glycol.
6. The composition of 5, wherein the hydrophobic segment comprises or is derived from poly(hexamethylene-alt-serinol) (PHS)
7. The composition of 1, wherein the reverse thermal gel comprises a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-functionalized polymer, acrylamide-based copolymer, and acrylonitrile-based copolymer compounds.
8. The composition of 7, wherein the backbone polymer comprises esterified poly[hexamethylene-alt-(serinol urea)].
9. The composition of 7, wherein the backbone polymer comprises a diol compound, wherein the diol compound comprises an amino-substituted or N-substituted serinol in which the nitrogen atom is attached to one of a hydrogen, a protective group or an active agent.
10. The composition of 7, wherein the graft polymer comprises poly(N-isopropylacrylamide).
11. The composition of 9, wherein the diol is an amino-substituted or N-substituted serinol, wherein the N is attached to one of a hydrogen, a protective group, or an active agent.
12. The composition of 9, wherein the N of the N-substituted serinol is —NHR in which R is an amine protective group.
13. The composition of 12, wherein R is selected from the group consisting of carbobenzyloxy; p-methoxyphenyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl.
14. The composition of 12, wherein R is tert-butyloxycarbonyl.
15. The composition of 12, wherein the diol comprises one or more ester groups.
16. The composition of 9, wherein the diol is a reaction product of a cyclic anhydride and a diol comprising one or more pendant active groups, blocked active groups or active agents.
17. The composition of 9, wherein the diol is the reaction product of succinic anhydride and the diol is an N-substituted serinol in which the N is attached to one of a hydrogen, a protective group, or an active agent.
18. The composition of 9, in which the diol comprises a pendant amino group or an amine.
19. The composition of 1, wherein the composition is a sol or a solution at room temperature, and form gels at about 30° C.-about 40° C., or at about 37° C. under physiological conditions.
20. The composition of 1, wherein the composition becomes a gel at about 35° C.-about 40° C.
21. An applicator such as a syringe, a tube, a dropper, comprising the composition of any one of 1-20.
22. A method of maintaining intravaginal pH at a pH of about 3.0-about 5.0, comprising administering an effective amount of the composition of any one of 1-20 vaginally thereby maintaining an intravaginal pH of about 3.0-about 5.0.
23. A method of female contraception, comprising administering a therapeutically effective amount of the composition of any one of 1-20 vaginally to a female in need thereof.
24. A method of prophylaxis or treatment of vaginal infection or urinary tract infection, comprising administering a therapeutically effective amount of a composition of any one of 1-20 to a patient in need thereof.
25. The composition of any one of 1-20 for female contraception or prophylaxis or treatment of vaginal infection or urinary tract infection.
26. A pharmaceutically acceptable composition comprising: an (a) A-B-A type block copolymer, wherein A comprises a polyethylene glycol, and B comprises a polyurethane; a B-A-B type block copolymer, wherein A comprises polyethylene glycol, and B comprises a polyhdroxyalkanoate, such as a co polymer of 2 hydroxyalkanoates, preferably, PLGA or a lactic acid-glycolic copolymer, an (ESHU); a (PEG-PACU) wherein the polyurethane excludes a amine functionality which is protonated at pH 3-5; a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxydhylmethacrylate, poly(N-acryloylglycinamide), ureido-functionalized polymer, acrylamide-based copolymer, and acrylonitrile-based copolymer compounds; a copolymer having a hydrophobic segment and one or more hydrophilic segments, wherein the hydrophobic segment and the one or more hydrophilic segments are capable of self assembly into a micelle; (b) one or more acidic pH adjustor, and (c) optionally one or more pharmaceutically acceptable excipients.
27. The composition of 26, wherein the composition excludes a poloamer.
28. The composition of 26, wherein the composition comprises a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-functionalized polymer, acrylamide-based copolymer, and acrylonitrile-based copolymer compounds.
29. The composition of 26, wherein the backbone polymer comprises esterified poly[hexamethylene-alt-(serinol; urea)].
30. The composition of 26, wherein the backbone polymer comprises a diol compound, wherein the diol compound comprises an amino-substituted or N-substituted serinol in which the nitrogen atom is attached to one of a hydrogen, a protective group or an active agent.
31. The composition of 26, wherein the composition comprises: a copolymer comprising a hydrophobic segment and one or more hydrophilic segments wherein the hydrophobic segment and one or more hydrophilic segments self-assemble into, a micelle.
32. The composition of 26, wherein the composition comprises: a copolymer comprising a hydrophobic segment and one or more hydrophilic segments wherein the hydrophobic segment and one or more hydrophilic segments self-assemble, into a micelle, wherein the hydrophilic segment is derived from polyethylene glycol, and the hydrophobic segment comprises or is derived from poly(hexamethylene-alt-serinol) (PHS), and the hydrophobic segment has a molecular weight of about 75,000 to about 200,000 Da.
33. The composition of 30, wherein the diol is an amino-substituted or N-substituted serinol, wherein the N is attached to one of a hydrogen, a protective group, or an active agent.
34. The composition of 33, wherein the N of the N-substituted serinol is —NHR in which R is a protective group.
35. The composition of 34, wherein R is selected from the group consisting of carbobenzyloxy; p-methoxybenzyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl.
36. The composition of 34, wherein R is tit-butyloxycarbonyl.
37. The composition of 30, wherein the diol comprises one or more ester groups.
38. A composition of 26, which is a sol or a solution at room temperature, and forms a gel at about 35° C.-about 40° C., such as at about 37° C. under physiological conditions.
39. An applicator such as a syringe, a tube, a dropper, comprising the composition of any one of 26-38.
40. A method of maintaining intravaginal pH at a pH of about 3.0-about 5.0, comprising administering an effective amount of the composition of any one of 26-38 vaginally thereby maintaining an intravaginal pH of about 3.0-about 5.0.
41. A method of female contraception, comprising administering a therapeutically effective amount of the composition of any one of 26-38 vaginally to a female in need thereof.
42. A method of prophylaxis or treatment of vaginal infection or urinary tract infection, comprising administering a therapeutically effective amount of a composition of any one of 26-38 to a patient in need thereof.
43. The composition of any one of 26-38 for female contraception or for prophylaxis or treatment of vaginal infection or urinary tract infection.
44. The composition of 3, wherein the reverse thermal gel is a B-A-B type block copolymer, wherein A comprises polyethylene glycol, and B comprises PLGA or a lactic acid-glycolic acid copolymer.
45. The pharmaceutically acceptable composition of 26, wherein the reverse thermal gel is a B-A-B type block copolymer, wherein A comprises polyethylene glycol, and B comprises PLGA or a lactic acid-glycolic acid copolymer.
46. A method of maintaining intravaginal pH at a pH of about 3.0-about 5.0, comprising administering an effective amount of the composition of any one of 44-45 vaginally thereby maintaining an intravaginal pH of about 3.0-about 5.0.
47. A method of female contraception, comprising administering a therapeutically effective amount of the composition of any one of 44-45 vaginally to a female in need thereof.
48. A method of prophylaxis or treatment of vaginal infection or urinary tract infection, comprising administering a therapeutically effective amount of a composition of any one of 44-45 to a patient in need thereof.
49. The composition of any one of 44-45 for female contraception or prophylaxis or treatment of vaginal infection or urinary tract infection.

### EXAMPLES

These examples illustrate the invention without limiting it.

### Example 1

### Preparation of a Pharmaceutical Composition

150 mg of poloxamer 407 (Kolliphor® P 407) and 150 mg poloxamer 188 (Kolliphor® P 188) and optionally 20 mg DL-α-Tocopherol are mixed together with and this mixture is added to 610 mg pre-cooled water (about 5° C.) in a transparent glass beaker and stirred with a magnetic stirring bar for 24-48 hours at about 4° C.

Gels containing various percentage of Poloxamer 407 and 188, are prepared analogous to Example 1 with or without a pH adjustor such as lactic acid, citric acid, or a mixture thereof The pH of the acidic gels are maintained within pH 3-5.

### Example 2A

### Appearance and pH Value

The appearance of the pharmaceutical compositions is assessed by visual observation: homogeneity, phase separation. pH values of the pharmaceutical compositions (after 1:10 w/v dilution with distilled water) is detected either using a pH meter, a pH paper, or an indicator.

### Example 2B

### Visual Detection of Composition Viscosity

An inverted test tube method is used to determine composition viscosity. 5 ml of the respective pharmaceutical composition containing different amounts of gelators (such as poloxamers and other gelators utilized herein (w/o additional excipients)) are transferred to sealed or stoppered glass vials and placed in a thermostat (4° C.) water bath. Temperature is stepwise increased up to 37° C. and left to equilibrate for 10 minutes at each new setting. At predefined temperatures viscosity is visually detected by turning the glass tubes upside down and classifying the flow properties of the transparent pharmaceutical composition into four categories:

1: liquid like water

2: slightly viscous

3: viscous to highly viscous

4: semisolid.

### Example 2C

### Injectability of Compositions at Room Temperature

Injectability of compositions is assessed by aspirating of 1 ml of the respective pharmaceutical composition into a 1 ml syringe to which a 0.9×40 mm cannula is attached. The force necessary to fill the syringe through the cannula is assessed as

1: force comparable to aspiration of water

2: force comparable to aspiration of oil

3: strong forces required

### Example 2D

### Measurement of Gel Strength

Gel strength of selected compositions is assessed by the following procedure: 1 ml g of the respective pharmaceutical composition is poured into a 2 ml screw capped glass vial and gelation is induced by maintaining the pharmaceutical composition to 37° C. Gel formation is observed within several minutes, usually within 1 to 10 minutes. To ensure full gel formation samples are incubated at 37° C. for about 30 minutes. After gel formation, 1 ml of prewarmed water (37° C.) is added, the vials are screwed again and incubated in the water bath with gentle shaking at 37° C. (30 rpm). The gel strength is determined as the time necessary for dissolution of the gel.

Pharmaceutical compositions showing desirable physical properties (e.g., 15% or 20% Poloxamer 407) according to thermo sensitive behavior are noted. Gels tested using 18% of Poloxamer 407 in combination with different concentrations of Poloxamer 188 (5%, 10%, 15%, 18%. Pharmaceutical compositions with 18% Poloxamer 407 and either 5% or 10% of Poloxamer 188 are determined. Pharmaceutical compositions that are liquid or in sol state at 4° C., slightly viscous at 22° C. and semisolid or solid at 37° C. are noted. In another batch, pharmaceutical compositions that are liquid or in sol state at higher temperature and semisolid or solid at about 37° C. or higher are noted.

### Example 3

A pharmaceutically acceptable aqueous sol including a reverse thermal gelator-PLGA-PEG-PLGA copolymer maintained at pH 3.5-4.5 is applied intravaginally to a female subject. The sol undergoes a sol to gel transformation once applied. The intravaginal pH is measured e.g. by holding a piece of pH paper against the wall of the vagina for a few seconds, and then comparing the color of the pH paper to the color on the chart provided with the test kit. The number on the chart for the color that best matches the color on the pH paper is the vaginal pH number.

What is claimed is:

1. A pharmaceutically acceptable composition comprising a reverse thermal gel, one or more acidic pH adjustor, and optionally one or more pharmaceutically acceptable excipients.

2. The composition of claim 1, wherein the one or more acidic pH adjustor maintains a pH of about 3-5.

3. The composition of claim 1, wherein the reverse thermal gel is: an A-B-A type block copolymer, wherein A comprises a polyethylene glycol, and B comprises a polyurethane; a B-A-B type block copolymer, wherein A comprises polyethylene glycol, and B comprises a polyhdroxyalkanoate, such as a co polymer of 2 hydroxyalkanoates, preferably, PLGA or a lactic acid-glycolic copolymer; an (ESHU); or a (PEG-PACU) wherein the polyurethane excludes an amine functionality which is protonated at pH 3-5.

4. The composition of claim 1, wherein the reverse thermal gel comprises a copolymer having a hydrophobic segment and one or more hydrophilic segments.

5. The composition of claim 4, wherein the hydrophilic segment comprises or is derived from polyethylene glycol.

6. The composition of claim 1, wherein the composition is a sol or a solution at room temperature, and form gels at about 30° C.-about 40° C., or at about 37° C. under physiological conditions.

7. The composition of claim 1, wherein the composition becomes a gel at about 35° C.-about 40° C.

8. An applicator such as a syringe, a tube, a dropper, comprising the composition of claim 1.

9. A method of maintaining intravaginal pH at a pH of about 3.0-about 5.0, comprising administering an effective amount of the composition of claim 1 vaginally thereby maintaining an intravaginal pH of about 3.0-about 5.0.

10. A method of female contraception, comprising administering a therapeutically effective amount of the composition of claim 1 vaginally to a female in need thereof.

11. A method of prophylaxis or treatment of vaginal infection or urinary tract infection, comprising administering a therapeutically effective amount of a composition of claim 1 to a patient in need thereof.

12. A pharmaceutically acceptable composition comprising: an (a) A-B-A type block copolymer, wherein A comprises a polyethylene glycol, and B comprises a polyurethane; a B-A-B type block copolymer, wherein A comprises polyethylene glycol, and B comprises a polyhdroxyalkanoate, such as a co polymer of 2 hydroxyalkanoates, preferably, PLGA or a lactic acid-glycolic copolymer; an (ESHU); a (PEG-PACU) wherein the polyurethane excludes a amine functionality which is protonated at pH 3-5; a grafted copolymer on a backbone polymer, wherein the backbone polymer comprises poly[hexamethylene-alt-(serinol urea)] (PHSU), and the graft copolymer is selected from the group consisting of poly(N-isopropylacrylamide) (PNIPAAm), hydroxypropylcellulose, poly(vinylcaprolactame), polyethylene oxide, polyvinylmethylether, polyhydroxyethylmethacrylate, poly(N-acryloylglycinamide), ureido-fimctionalized polymer, acrylamide-based copolymer, and acrylonitrile-based copolymer compounds; a copolymer having a hydrophobic segment and one or more hydrophilic segments, wherein the hydrophobic segment and the one or more hydrophilic segments are capable of self assembly into a micelle; (b) one or more acidic pH adjustor, and (c) optionally one or more pharmaceutically acceptable excipients.

13. The composition of claim 12, wherein the composition excludes a poloxamer.

14. The composition of claim 12, wherein the composition comprises: a copolymer comprising a hydrophobic segment and one or more hydrophilic segments wherein the hydrophobic segment and one or more hydrophilic segments self-assemble into a micelle.

15. A composition of claim 12, which is a sol or a solution at room temperature, and forms a gel at about 35° C.-about 40° C., such as at about 37° C. under physiological conditions.

16. An applicator such as a syringe, a tube, a dropper, comprising the composition of claim 12.

17. A method of maintaining intravaginal pH at a pH of about 3.0-about 5.0, comprising administering an effective amount of the composition of claim 12 vaginally thereby maintaining an intravaginal pH of about 3.0-about 5.0.

18. A method of female contraception, comprising administering a therapeutically effective amount of the composition of claim 12 vaginally to a female in need thereof.

19. A method of prophylaxis or treatment of vaginal infection or urinary tract infection, comprising administering a therapeutically effective amount of a composition of claim 12 to a patient in need thereof.

20. The composition of claim 3, wherein the reverse thermal gel is a B-A-B type block copolymer, wherein A comprises polyethylene glycol, and B comprises PLGA or a lactic acid-glycolic acid copolymer.
